(19) United States
(12) Patent Application Publication
(10) Pub. No.: US 2023/0192788 A1
(43) Pub. Date: Jun. 22, 2023

(54) KERATIN HYDROLYSATE HIGH IN FREE AMINO ACIDS AND HIGH IN FREE TYROSINE, METHOD FOR OBTAINING AND USING SAME FOR ANIMAL FEED AND PLANT NUTRITION

(71) Applicant: BRETAGNE CHIMIE FINE, Pleucadeuc (FR)

(72) Inventors: Etienne BROIN, LYON (FR); Joel DUPERRAY, LYON (FR); Jean-Philippe SOULARD, LYON (FR); Renaud SERGHERAERT, LYON (FR)

(73) Assignee: BRETAGNE CHIMIE FINE, Pleucadeuc (FR)

(21) Appl. No.: 17/923,334

(22) PCT Filed: May 25, 2021
(86) PCT No.: PCT/EP2021/061812

§ 371 Date: Nov. 4, 2022

(30) Foreign Application Priority Data
May 7, 2020 (FR) 2004564

(51) Int. Cl. C07K 14/47; C07K 1/12; A23K 20/142; A23K 10/26; A01N 63/50

(52) U.S. Cl. CPC C07K 14/4741; C07K 1/12; A23K 20/142; A23K 10/26; A01N 63/50

(57) ABSTRACT

A keratin hydrolysate including at least 88% by weight of free amino acids relative to the total weight of the amino acids of the hydrolysate and including a content of free tyrosine between 2% and 4% by weight relative to the total weight of the free amino acids of the hydrolysate. Also, a method for preparing the hydrolysate including the following steps: acid hydrolysis, a pH adjustment and a desalination. Also, the use of the hydrolysate for pet food, aquaculture food, and plant biostimulants.

This invention relates to the field of hydrolysates with high contents of free amino acids and to their uses in animal feed, in particular feline and canine, in aquaculture and agriculture, notably as plant biostimulants.

Compositions based on amino acids are used in a range of fields such as nutraceuticals, cosmetics, plant, animal and human nutrition, and for very different and specific applications in each of these fields. These include the uses aimed at the growth and shine of hair in humans, as well as the uses aimed at supplying free amino acids constituting a specific protein source in animal feed, particularly in aquaculture and canine and feline nutrition.

One of the ways to obtain a composition based on amino acids is to produce a hydrolyzate of keratin materials.

Natural keratin materials is essentially made up of polypeptides of high molecular weight and with a highly cross-linked structure, making it not easily accessible to enzymes. This natural keratin material is not easily digestible. It is known, however, that the hydrolysis of keratin materials into amino acids enhances their digestibility.

The keratin hydrolysates offered for sale in particular as food supplements, ingredients for formulating animal nutrition recipes, or raw materials for animal feed are generally obtained through very partial hydrolysis. These hydrolysates generally feature a high molecular mass due to the presence of high levels of so-called “linked” amino acids forming peptides and polypeptides. The molecular weight of commercial compositions typically ranges from 5.000 to 50.000 daltons. These keratin hydrolysates are relatively indigestible and contain little to no free amino acids. Indeed, it is technically complicated and expensive to obtain a keratin hydrolyzate with a very high level of free amino acids on an industrial level.

In addition, excessive hydrolysis presents a risk of amino acid denaturation and destruction.

Patent application WO2019/043128 outlines a keratin hydrolyzate with high levels of free amino acids, obtained by implementing a process in which the hydrolysis step is followed by a tyrosine extraction step. This hydrolyzate has the advantages pertaining to high levels of free amino acids, in particular good digestibility. This hydrolyzate, however, does not include all the amino acids generally present after acid hydrolysis, in particular the levels of cysteine and tyrosine are low. Nonetheless, obtaining a hydrolyzate that can be used in a complete and balanced food, without adding additional amino acids, is a property that is generally sought after.

Furthermore, the hydrolysates obtained through chemical hydrolysis, such as acid hydrolysis, are in salted form, whereas most uses, in particular in animal feed and in biostimulation, require desalted hydrolysates. Still, desalination processes generally lead to a loss of a significant quantity of certain amino acids, in particular amino acids which precipitate at the ending of the neutralization stage. In fact, desalination can only be carried out on a filtered solution. The yields of the hydrolysates obtained according to the methods of the prior art, therefore, are not always satisfactory.

Surprisingly and to further advantage, the inventors of this invention have succeeded in overcoming the problems of the prior art by the implementation of a process making it possible to obtain a desalted keratin hydrolyzate, having a high level of acids free amino acids and comprising all the amino acids generally present after acid hydrolysis. In addition, the amino acid profile of the hydrolyzate based on this invention is close to that of the original keratin material.

The use of the hydrolyzate according to this invention, therefore, makes it possible to do away with the great majority of the addition of additional amino acids, among those present in the original keratin material.

The free amino acids obtained per the invention are not damaged or denatured, in particular the amino acids among the most difficult to release in a free form during the hydrolysis process such as valine, leucine, and isoleucine.

Furthermore, the process according to the invention makes it possible to obtain a good yield of amino acids, i.e. a good ratio between the total of the total amino acids of the hydrolyzate according to the invention and the keratin material starting point, since all the phases resulting from the various stages of the process are recovered and treated so as to extract the maximum, that is, practically all the amino acids.

The subject of this invention is a keratin hydrolyzate comprising at least 88%, preferably at least 90%, by weight of free amino acids relative to the total weight of the amino acids of the hydrolyzate, the said hydrolyzate comprising free tyrosine in a content ranging from 2 to 4%, preferably 2.5 to 3.5% by weight relative to the total weight of the free amino acids of the hydrolyzate.

Advantageously, the said hydrolyzate comprises at least 90%, preferably at least 93%, and preferably at least 95% of cystine in free form by weight relative to the total weight of cystine in the hydrolyzate.

In a preferred embodiment, the said hydrolyzate is desalted, i.e. it comprises less than 11%, preferably less than 7% by weight of salts relative to the total weight of the hydrolyzate, the salts being chosen from sodium chloride, sodium sulphate, sodium phosphate, potassium chloride, potassium sulphate and potassium phosphate, preferably sodium chloride.

A second object of this invention relates to a process for preparing the keratin hydrolyzate according to the invention from an animal keratin material, preferably poultry, comprising at least the following steps, in the order outlined below:
subjecting the keratin material to at least one chemical hydrolysis by means of an acid under conditions capable of obtaining a hydrolyzate comprising at least 88% by weight of free amino acids relative to the total weight of the amino acids of the hydrolyzate, the rest of the amino acids of the hydrolyzate being in the form of peptides having a molecular mass less than or equal to 800 Dalton;
subjecting the hydrolyzate to a step of adjusting the pH to a value ranging from 3 to 5, preferably ranging from 4 to 5 and recovering the precipitate and the liquid phase;
separating the precipitate and the liquid phase, preferably through spinning;
subjecting the precipitate to at least one washing with water until a desalted precipitate is obtained comprising less than 1% of salts by weight relative to the total weight of the precipitate, and recovering the desalted precipitate on the one hand, and washing water on the other one,
gathering the washing water and the liquid phase to obtain a solution and proceed with desalinating this solution by electrodialysis to obtain a desalinated solution,
adding the desalted precipitate to the said desalted solution,
recovering the desalted suspended matter obtained.

In a preferred variant, the suspended matter obtained is dried and the solid obtained at the end of the drying recovered.

The means for implementing the process according to the invention have the advantage of being simple: the invention can be implemented with a device using means commonly used in industry such as reactors, spinners, and atomization towers. Several steps can be performed in the same enclosure, furthermore the gathering of the different phases is carried out easily without notable problems.

This invention further relates to the use of the keratin hydrolyzate in animal feed, in particular feline, canine, in aquaculture or in agriculture. This invention relates more particularly to the use of the hydrolyzate according to the invention or prepared according to the invention as a component of a product chosen from foods for animal feed for pets, foods for the aquaculture, and plant biostimulants.

Other aspects, advantages, and properties of the invention will emerge clearly from the description, in particular from the examples, and from the drawings which follow, by way of non-exhaustive indication.

### DETAILED DESCRIPTION

Advantageously, this hydrolyzate is obtained from natural, animal, keratin materials in particular from poultry, favorably from poultry feathers. Speaking of poultry, hens, in particular laying hens, chickens, turkeys, ducks, geese, etc may be cited. The natural keratin materials may also be chosen from animal hair, in particular pig bristles, animal hooves, animal nails.

In particular, the hydrolyzate according to the invention is not obtained from human keratin such as hair.

As already mentioned, the hydrolyzate according to the invention comprises at least 88%, preferably 90% by weight of free amino acids relative to the total weight of the amino acids of the hydrolyzate.

Advantageously, the total amino acid content of the hydrolyzate according to the invention ranges from 40% to 95%, preferably 45% to 93% by weight relative to the total weight of the hydrolyzate, the hydrolyzate further comprising mineral matter and water.

In addition, the invention-based hydrolyzate features free branched amino acids: undenatured valine, leucine and isoleucine. However, these branched amino acids are known to be more difficult to release under identical conditions of use.

As already mentioned, the hydrolyzate according to the invention comprises at least 90%, preferably at least 93% and preferably at least 95% of cystine in free form by weight relative to the total weight of cystine in the hydrolyzate.

In a preferred variant, hydrolyzate according to the invention comprises the following: at least 95%, preferably 100%, of aspartic acid in free form by weight relative to the total weight of aspartic acid in the hydrolyzate;

at least 95%, preferably 100%, of threonine in free form by weight relative to the total weight of threonine in the hydrolyzate;

at least 95%, preferably 100%, of serine in the free form by weight relative to the total weight of serine in the hydrolyzate;

at least 93%, preferably 95% or more, of glutamic acid in the free form by weight relative to the total weight of glutamic acid in the hydrolyzate;

at least 90%, preferably 93% or more, of glycine in the free form by weight relative to the total weight of glycine in the hydrolyzate;

at least 90%, preferably 93% or more, of alanine in the free form by weight relative to the total weight of alanine in the hydrolyzate;

at least 90%, preferably 93% or more, of phenylalanine in the free form by weight relative to the total weight of phenylalanine in the hydrolyzate;

at least 93%, preferably 95% or more, of proline in free form by weight relative to the total weight of proline in the hydrolyzate.

Furthermore, at least 90% by weight of the amino acids of the hydrolyzate feature a molecular mass less than or equal to 250 daltons, preferably less than or equal to 240 daltons. As a result, this hydrolyzate can be used to prepare complete feedstuffs for animal feed with hypoallergenic or even anallergic properties.

The obtained hydrolyzate is advantageously desalted, i.e. it comprises less than 11%, preferably less than 7% by weight of salts relative to the total weight of the hydrolyzate, the salts being chosen from chloride of sodium, sodium sulphate, sodium phosphate, potassium chloride, potassium sulphate and potassium phosphate, preferably sodium chloride (NaCl).

Determining the percentage of salts falls within the competence of a skilled person. Preferably, the percentage of salts is determined by anion dosage. In particular, the chloride ions are determined by a potentiometric dosage, using 0.1 N silver nitrate, followed by a combined Ag/AgCl electrode; the phosphate ions are determined by spectrophotometric determination of a phosphomolybdic complex as per the ISO 6878 standard and the sulphates by gravimetric determination by adding a barium salt based on the ISO 2480:1972 standard.

The anion dosage can be supplemented further by cation dosage, in general the dosage of sodium and potassium is taken by spectrophotometry by flame ionization based on the ISO 9964-2:1993 standard.

The salt(s) content depends on the quality of washing of the precipitate and the degree to which the electrodialysis is continued. It is within the competence of a skilled person to adjust the parameters of these stages, in particular to adapt their durations, based on the required salt(s) content.

“dry hydrolyzate” or “dried hydrolyzate”, as defined in this invention, means a hydrolyzate containing less than 5% by weight of water. The water weight of the hydrolyzate is measured using an infrared thermobalance.

Preferably, the hydrolyzate according to the invention comprises the following free amino acids, by weight, relative to the total weight of the free amino acids of the hydrolyzate:

aspartic acid in a content ranging from 6.00 to 10.00%, preferably ranging from 7.00 to 9.00%, and, more preferably, 7.83% by weight;

threonine in a content ranging from 3.00 to 7.00%, preferably ranging from 4.00 to 6.00%, and, more preferably, 4.93% by weight;

serine in a content ranging from 11.00 to 15.00%, preferably ranging from 12.00 to 14.00%, and, more preferably, 12.88% by weight;

glutamic acid in a content ranging from 8.50 to 12.50%, preferably ranging from 9.50 to 11.50%, and, more preferably, 10.47% by weight;

glycine in a content ranging from 6.50 to 10.50%, preferably ranging from 7.50 to 9.50%, and, more preferably, 8.56% by weight;

alanine in a content ranging from 3.00 to 7.00%, preferably ranging from 4.00 to 6.00%, and, more preferably, 5.04% by weight;

valine in a content ranging from 3.50 to 7.50%, preferably ranging from 4.50 to 6.50%, and, more preferably, 5.61% by weight;

cystine in a content ranging from 4.00 to 8.00%, preferably ranging from 5.00 to 7.00%, and, more preferably, 5.80% by weight;

methionine in a content ranging from 0.10 to 2.00%, preferably ranging from 0.20 to 1.00%, and, more preferably, 0.57% by weight;

isoleucine in a content of 1.50 to 5.50%, preferably ranging from 2.50 to 4.50%, and, more preferably, 3.50% by weight;

leucine in a content ranging from 6.00 to 10.00%, preferably ranging from 7.00 to 9.00%, and, more preferably, 7.77% by weight;

tyrosine in a content ranging from 2.50 to 3.50%, preferably 3.00 to 3.50% and, more preferably, 3.15% by weight;

phenylalanine in a content ranging from 3.00 to 7.00%, preferably ranging from 4.00 to 6.00%, and, more preferably, 5.08% by weight;

lysine in a content ranging from 0.50 to 3.00%, preferably ranging from 1.00 to 2.00%, and, more preferably, 1.66% by weight;

histidine in a content ranging from 0.10 to 2.00%, preferably ranging from 0.20 to 1.00%, and, more preferably, 0.74% by weight;

arginine in a content ranging from 4.00 to 8.00%, preferably ranging from 5.00 to 7.00%, and, more preferably, 5.82% by weight;

proline in a content ranging from 8.50 to 12.50%, preferably ranging from 9.50 to 11.50%, and, more preferably, 10.59% by weight.

Another advantage of the invention-based keratin hydrolyzate is that its amino acid profile is close to that of the original keratin material. Indeed, except for tryptophan destroyed in acid hydrolysis, the 17 amino acids present in the original keratin material appear in free form in the final hydrolyzate as well.

The invention-based keratin hydrolyzate, therefore, comprises 17 amino acids, and for each of the amino acids, the percentage variation between the weight of the free amino acid in the hydrolyzate and the weight of this amino acid in the keratin material starting point is less than 20% in absolute value, advantageously for 15 of these amino acids, the said variation is less than 10% in absolute value.

The variation, in percentage, for an amino acid corresponds to the ratio of the absolute value of the difference between the weight of the amino acid in the keratin material and the weight of the free amino acid in the hydrolyzate on the weight of the amino acid in the keratin material multiplied by 100, i.e. the following formula:

(I weight of the amino acid in the keratin material−weight of the free amino acid in the hydrolyzate|/weight of the amino acid in the keratin material)×100.

Another advantage of the hydrolyzate according to the invention is that it is very digestible. Moreover, it is recognized to be food grade. The hydrolyzate according to the invention features a true digestibility of its protein fraction of at least 98%. This value is very close to the maximum possible (100%).

The digestibility is measured in vivo according to the method described by Z. M. Larbier, A. M. Chagneau, and M. Lessire in “Effect of protein intake on true digestibility of amino acids in rapeseed meals for adult roosters force fed with moistened feed.” Animal Feed Science and Technology. 34 (1991) 255-260.

Process

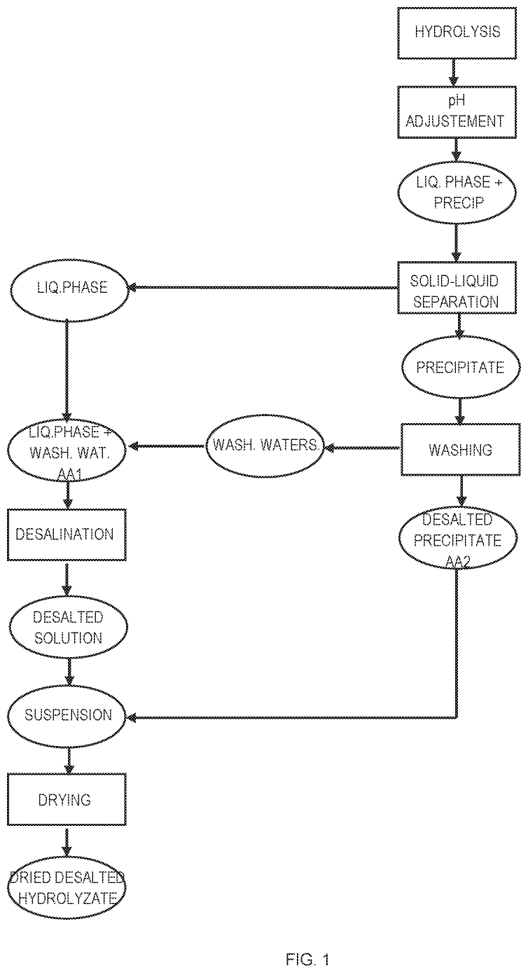

FIG. 1 is a diagram showing the main steps of the process according to the invention described below as well as the phases obtained at the end of these different steps. The steps are presented in rectangles and the phases in ellipses. Following the diagram in FIG. 1, the process includes hydrolysis followed by a pH adjustment step leading to a liquid phase and a precipitate that are subjected to a solid-liquid separation step. The precipitate is then subjected to washing to yield the desalted precipitate (AA2). The liquid phase and the washing waters are combined and this solution (AA1) is subjected to a desalination stage yielding a desalinated solution. Then the desalted solution and the desalted precipitate (AA2) are combined and the obtained suspended matter is subjected to drying leading to the obtaining of the dried desalted hydrolyzate.

Acid Hydrolysis

The process for preparing the keratin hydrolyzate according to the invention implements at least one chemical hydrolysis by means of an acid in conditions suitable for obtaining a hydrolyzate comprising at least 88% by weight of free amino acids with respect to the total weight of the amino acids of the hydrolyzate, the rest of the amino acids of the hydrolyzate being in the form of small peptides, i.e. having a molecular mass less than or equal to 800 Dalton.

The percentage of small peptides in the hydrolyzate according to the invention generally ranges from 5 to 12% by weight relative to the total hydrolyzate weight.

Indeed, the hydrolysis not being total, the percentage of small peptides in the hydrolyzate is not zero, and is 12% by weight at most.

The chemical hydrolysis of the keratin is performed using an acid, preferably a strong acid chosen from hydrochloric, phosphoric and sulfuric acids, preferably hydrochloric acid. The concentration of acid, preferably hydrochloric one, should range from 14 to 34% by weight.

Preferably, the acid/keratin material weight ratio, in particular the acid/feather weight ratio, ranges from 2 to 5.

The chemical hydrolysis is generally performed for a period ranging from 1 hour to 24 hours, preferably ranging from 6 to 20 hours at a temperature ranging from 100 to 115° C.

Based on a specific variant, the chemical hydrolysis is carried out in two stages: a first chemical hydrolysis performed at a temperature ranging from 60 to 80° C. for a period ranging from 4 to 5 hours. Then a second chemical hydrolysis is performed at a temperature ranging from 100 to 115° C. for a period of 5 to 8 hours.

In addition, the two hydrolyses can be performed without an intermediate pause step or by carrying out an intermediate pause step of between 1 hour and 7 days.

More precisely, the first chemical hydrolysis is performed at 72° C. for 4.5 hours and the second chemical hydrolysis is carried out at 107° C. for 6 hours, with an intermediate pause of 24 to 80 hours taken between the two chemical hydrolyses.

Advantageously, when a fatty part floats on the surface of the hydrolyzate, this supernatant is eliminated.

pH Adjustment

Chemical hydrolysis, performed in one or more stages, is followed by a pH adjustment stage. The hydrolyzate is brought to a pH having a value ranging from 3 to 5, preferably from 4 to 5. This step is performed by adding a base chosen from sodium hydroxide and potassium hydroxide, preferably sodium hydroxide. This step is a classic one, which implementation falls within the competence of a skilled person.

This step also features the effect of precipitating, at least partially, the less soluble amino acids, in particular cystine, tyrosine, leucine, and isoleucine. These less soluble amino acids form the precipitate, the other amino acids remain in solution and form, with the salt formed and the water, the liquid phase.

Solid-Liquid Separation—Dewatering

The pH adjustment step is followed by a step of separating the precipitate from the liquid phase. The separation step can be carried out by implementing any solid-liquid separation technique, in particular by applying a centrifugal force or by pressing, using a filter press in particular. Based on a preferred variant, the separation step is a dewatering. The dewatering stage is advantageously performed by applying a centrifugal force through an approximately 1000-rpm rotation. This method, known to those skilled in the art for performing a solid-liquid separation, consists in eliminating the liquid phase by the effect of centrifugal force, while maintaining the precipitate (solid fraction) on a cloth.

Washing

The precipitate, spun, recovered, preferably on the cloth of the wringer, is then washed with water until a desalted precipitate with a salt content of less than 1% by weight, relative to the total weight of the precipitate, is obtained. The term ‘salts’ implies sodium chloride, sodium sulphate, sodium phosphate, potassium chloride, potassium sulphate and potassium phosphate, preferably sodium chloride (NaCl).

During the washing stage, the water which passes over the precipitate in place on the fabric entrains, by solubilization, the salts present in the said precipitate as well as some amino acids, while leaving the most insoluble amino acids in solid form.

At the end of the washing stage, the water content of the precipitate ranges from 50 to 60% by weight relative to the total weight of the precipitate.

The desalted precipitate mainly comprises the following amino acids: cystine, tyrosine and leucine, it also comprises valine, isoleucine, and phenylalanine.

The possibility of performing the spin-drying and the washing in the same enclosure, in particular in the wringer, contributes to simplifying the means of implementing the process according to the invention.

The washing waters are recovered and added to the liquid phase obtained at the end of the spin cycle to form a solution of salty amino acids.

Desalination

The salty amino acid solution is subjected to the desalination stage through electrodialysis. This salty amino acid solution includes all the amino acids, yet cystine and tyrosine are present in it in very small quantities.

The desalination stage aims to eliminate the salts, in particular the sodium chloride formed during the pH adjustment stage by adding sodium hydroxide to the hydrochloric acid used through the hydrolysis stage. This desalination step is performed by means of electrodialysis. Electrodialysis is conventionally performed by opposing pure water to the solution to be desalinated, the two solutions circulating separately and alternately between anionic and cationic membranes an electric current is applied on. At the end of the desalination stage, the salt content of the solution is less than 1% by weight of salt relative to the total solution weight.

Phase Combination

Following the desalination stage, the desalted precipitate obtained at the end of the water washing step is introduced into the desalinated solution, thus forming suspended matter.

Surprisingly and advantageously, the inventors have shown that all the amino acids resulting from acid hydrolysis are present. In addition, the amino acid profile of the hydrolyzate according to the invention is close to that of the original keratin material.

Preferably, the suspended matter obtained is dried and the solid obtained following the drying stage is recovered. The hydrolyzate according to the invention is preferably in dry form, it comprises less than 5% by weight of water relative to the total weight of the hydrolyzate.

The hydrolyzate in dry form comprises less than 11%, preferably less than 7% by weight of salts relative to the total hydrolyzate weight.

The weight of total amino acids recovered from the hydrolyzate in dry form is at least equal to 80% of the weight of the total amino acids contained in the keratin material involved in the hydrolysis, preferably at least 84%.

Uses

As already mentioned, this invention relates to the use of the hydrolyzate in animal nutrition, in particular feline, canine, in aquaculture, or in agriculture.

This invention relates more particularly to the use of the hydrolyzate according to the invention or prepared according to the invention as a component of a product chosen from foods for animal feed for pets, foods for the aquaculture, and plant biostimulants.

According to a first variant, the invention is aimed at the use, orally, of the keratin hydrolyzate according to the invention, or obtained by the preparation process according to the invention, as a raw material for animal feed.

The invention also relates to a raw material comprising a hydrolyzate according to the invention without ingredient additions.

The term “raw material” means any product of plant or animal origin, in its natural state, fresh or preserved, and derived from their industrial processing, as well as any organic or inorganic substances, whether or not including additives, which are intended for use in animal feeding orally, either directly as such or after processing, for the preparation of compound feedingstuffs, or as carriers for premixtures (Directive 96/25/EC of the Council of Apr. 29, 1996).

The raw material according to the invention is a mixture of amino acids intended to be incorporated into a complete and balanced foodstuff in animals or to be used as a food supplement in humans. It is therefore intended for oral administration in terrestrial and/or marine animals and/or in humans. This raw material does not fall into the therapeutic category.

This invention relates more particularly to the use of the hydrolyzate, in animal feed and, more particularly, as a raw material source of free amino acids, making it possible to dispense with food proteins of vegetable and/or animal origin of complex molecular structure and of high molecular weight.

Formulating the raw material for animal feed in accordance with the invention implements conventional processes, which form part of the general skills of those skilled in the art.

As already mentioned, this invention further relates to a complete feed for animal feeding comprising from 0.25 to 40% by weight of the composition or preferably of the hydrolyzate according to the invention relative to the total weight of the said complete feed.

The complete feed for animal feeding in accordance with the invention can be formulated with the excipients usually used in compositions intended for the oral route, in particular humectants, thickeners, texturizing agents, flavoring agents, coating agents, preservatives, antioxidants, colorants, plant extracts, non-protein ingredients such as starches, vegetable fibers, minerals, and vitamins.

Of course, those skilled in the art will take care to choose these excipients so as not to alter the properties of the complete feed for animal feeding.

The complete feed for animal feeding according to the invention can be formulated based on one of the following presentations: a kibble, a dragee, a tablet, a soft or hard capsule, or even a suspension, a solution, a gel, a dry preparation containing less than 15% by weight of water, or a wet preparation comprising at least 50% by weight of water and a maximum of 85% by weight of water.

Formulating the complete feed for animal feeding according to the invention implements conventional processes, which form part of the general skills of those skilled in the art.

The invention also relates to the use of the composition according to the invention or the hydrolyzate according to the invention to prepare a raw material or complete feed for animal feeding.

According to a second variant, this invention relates to the use of keratin hydrolyzate as an ingredient promoting palatability in feed for aquaculture, in particular for shrimp farming, in particular at the larval stage and up to at the growth stage.

According to a third variant, this invention targets the use of keratin hydrolyzate as a plant biostimulant. The hydrolyzate according to the invention can be used on the different parts of plants: seeds, leaves, flowers, and fruits.

Biostimulants are defined as substances and/or microorganisms which function, when applied to plants or the rhizosphere, lies in stimulating natural processes that foster/enhance the absorption or use of nutrients, tolerance to abiotic stresses, the quality or yield of the crop, independently of nutrient presence.

The hydrolyzate can be used further with an ingredient chosen from phytosanitary products, fertilizers, microorganisms, seaweed extracts, humic and fulvic acids, as well as minerals.

The following examples are intended to illustrate the invention without limiting its scope.

### EXAMPLES

The amino acids presented in Tables 1 to 3 are measured based on a method adapted from the 152/2009 EC regulation.

The amino acids are separated by chromatography (HPLC) with an ion exchange column and assayed by reaction with ninhydrin and photometric detection at 570 nm.

### Example 1—Hydrolyzate

Hydrolyzate Preparation

Hydrolysis

9000 kg of poultry feathers containing 50% dry matter are introduced into a 55.000 liter reactor/hydrolyser. Chemical hydrolysis is performed by adding 18.000 liters of hydrochloric acid (23%), hydrolysis is carried out at 72° C. for 4.5 hours. The resulting product is stored for 48 hours, thus enabling its temperature to evolve naturally to room temperature. A second chemical hydrolysis is then performed by heating at 107° C. for 6 hours without adding acid. The hydrolyzate obtained comprises 88% by weight of free amino acids, the rest of the amino acids of the hydrolyzate being in the form of small peptides having a molecular mass less than or equal to 800 Dalton.

Purification

The hydrolyzate is then decanted to eliminate the fat left by the keratin material which floats on the surface of the aqueous phase. The hydrochloric acid introduced in excess during the hydrolysis step is removed. 8000 kg of concentrate are recovered. 4500 kg of water are then added to obtain 12500 kg of diluted concentrate.

pH Adjustment

30.5% sodium hydroxide is added to the hydrolyzate to bring the pH to a value between 4 and 5. When the sodium hydroxide is added, the less soluble amino acids, in particular cystine, tyrosine, leucine, and isoleucine precipitate, at least partially. The other amino acids remain in solution in full in the liquid phase.

Dewatering

The suspended matter is then placed in a wringer, to separate the precipitate (which remains on the canvas), and recover the liquid phase, corresponding to 17.000 kg, in a tank.

Washing

The precipitate remaining on the canvas is then washed by introducing 3000 kg of water directly into the wringer to remove the salt (NaCl). The washing waters, corresponding to 3440 kg, are sent to the tank which already contains the liquid phase resulting from the spinning, to yield 20440 kg of a solution called AA1. The 1560 kg of washed precipitate are recovered, which corresponds to product AA2.

The free amino acid composition of the AA1 solution is shown in Table 1

TABLE 1

| | % free amino acids |
|---|---|
| | in AA1 solution by |
| | weight relative to |
| | total free amino acids |
| Aspartic acid | 9.41% |
| Threonine | 5.90% |
| Serine | 15.40% |
| Glutamic acid | 12.46% |
| Glycine | 10.25% |
| Alanine | 5.88% |
| Valine | 6.16% |
| Cystine | 0.04% |
| Methionine | 0.42% |
| Isoleucine | 2.78% |
| Leucine | 4.01% |
| Tyrosine | 0.03% |
| Phenylalanine | 4.66% |
| Lysine | 1.99% |
| Histidine | 0.89% |
| Arginine | 7.00% |
| Profine | 12.72% |
| total | 100.00% |

AA1 features a dry matter content of 34.07%, measured by means of an infrared thermobalance, an NaCl content of 14.74% and a free amino acid content of 93.4% by weight relative to the weight total AA1 amino acids.

The 1560 kg of washed precipitate, containing less than 1% NaCl, are recovered. The dry matter content is 44% by weight by measurement using an infrared thermobalance. The free amino acid composition of the washed and dried precipitate is shown in Table 2.

TABLE 2

| | % free AA in washed precipitate (AA2) by |
|---|---|
| | weight relative to total free AA |
| Aspartic acid | 0.07% |
| Threonine | 0.14% |
| Serine | 0.28% |
| Glutamic acid | 0.49% |
| Glycine | 0.18% |
| Alanine | 0.79% |
| Valine | 2.49% |
| Cystine | 38.54% |
| Methionine | 1.08% |
| Isoleucine | 6.00% |
| Leucine | 22.26% |
| Tyrosine | 21.61% |
| Phenylalanine | 6.08% |
| Lysine | 0.00% |
| Histidine | 0.00% |
| Arginine | 0.00% |
| Proline | 0.01% |
| total | 100.00% |

The AA2 precipitate features a free amino acid content of 92.17% by weight relative to the total weight of the total amino acids of the AA2 precipitate.

Desalination

The AA1 solution (liquid phase from spin-drying and precipitate washing waters combined) weighing 20.440 kg is desalinated by electrodialysis against water, to yield approximately 14.300 kg of desalinated solution, containing less than 1% NaCl. The electrodialyzer used to perform the desalination consists of a double stack of 2×600 alternating anionic and cationic membranes, between which the solutions circulate, and traversed by a direct electric current.

Phase Combination

The washed precipitate corresponding to 1560 kg and the desalinated solution corresponding to 14.300 kg are combined to yield 15.860 kg of suspension, which is dried by atomization in a drying tower with an inlet temperature of 172° C. and an outlet of 80° C. and with a 2500-rpm sieving. About 3860 kg of powder are obtained, which composition is given in Table 3.

Table 3 presents, in the second column: the weight fraction of each FAA (free amino acids) of the hydrolyzate relative to the total FAA; in the third column: the weight fraction of each AA (amino acids) of the original keratin material relative to the total AA; in the fourth column, the variation in percentage, in absolute value, between the weight of the free amino acid in the hydrolyzate and the weight of this amino acid in the keratin material.

TABLE 3

| | | | Variation between |
|---|---|---|---|
| | | | FAA in the |
| | | | hydrolyzate and AA in |
| | FAA content in the | AA content in the | the original material in |
| | hydrolyzate (% of | original keratin material | % and in absolute |
| | total FAA) | (% of total AA) | value |
| Aspartic acid | 7.83% | 7.34% | 6.68% |
| Threonine | 4.93% | 4.58% | 7.64% |
| Serine | 12.88% | 13.20% | 2.42% |
| Glutamic acid | 10.47% | 10.44% | 0.29% |
| Glycine | 8.56% | 8.39% | 2.03% |
| Alanine | 5.04% | 4.78% | 5.44% |
| Valine | 5.61% | 5.19% | 8.09% |
| Cystine | 5.80% | 6.98% | 16.91% |
| Methionine | 0.57% | 0.54% | 5.56% |
| Isoleucine | 3.50% | 3.46% | 1.16% |
| Leucine | 7.77% | 7.75% | 0.26% |
| Tyrosine | 3.15% | 3.37% | 6.53% |
| Phenylalanine | 5.08% | 5.03% | 0.99% |
| Lysine | 1.66% | 1.64% | 1.22% |
| Histidine | 0.74% | 0.65% | 13.85% |
| Arginine | 5.82% | 6.13% | 5.06% |
| Proline | 10.59% | 10.54% | 0.47% |
| total | 100.00% | 100.00% | |

The obtained hydrolyzate has a dry matter content of 98.6%, an NaCl content of 4.7% and a free amino acid content of 91.11% by weight relative to the total weight of the total hydrolyzate amino acids.

The starting keratin material contains 93% of total amino acids on dry matter (4500 kg of dry matter), and the obtained hydrolyzate (3860 kg as is) contains 90.6% of total amino acids on as is, the yield of total amino acids is 83.6%.

In addition, the amino acid profile of the hydrolyzate according to the invention is close to that of the original keratin material. In fact, as presented in the fourth column, for each of the 17 amino acids, the percentage variation between the weight of the free amino acid in the hydrolyzate and the weight of this amino acid in the starting keratinous material is less than 20% in absolute value. Moreover, for 15 of them, this variation in weight is less than 10%.

### Example 2—Digestibility

Example 2—Digestibility The true digestibility of the proteins of the hydrolyzate according to the invention is very high since it equals 98.99% and therefore very close to the maximum value possible (100%). This value was obtained according to the following protocol on caecectomized cockerels, which is a reference model for measuring the bioavailability of proteins in the animal kingdom.

### Experimental Protocol

The digestibility measurements are taken on caecectomized adult cockerels housed in individual cages and fed outside the test period with a standard diet.

2 repetitions of 4 caecectomized cockerels are used. All the animals are fasted for 24 hours prior to ingesting a single meal of 80 g composed of 24 g of sample (hydrolysate) mixed with 56 g of sugar.

All faeces (=feces), including endogenous losses, are collected during the next 48 hours in two 24-hour periods to avoid their fermentation and possible deterioration.

These faeces, excluded from any contamination such as feathers for example, are carefully removed prior to being frozen (−80° C.).

The faeces are then freeze-dried in the oven, grouped together and mixed in 2 pools corresponding to the 2 replicates of 4 animals used for each of the 2 hydrolysates. The 2 pools are analyzed.

Nutritional analyzes (dry matter, crude protein (Dumas method ISO 16634-1:2008 standard) are carried out on the hydrolyzate, the cockerel faeces as well as on the endogenous losses. This data is used to calculate true protein digestibility.

For this value of true protein digestibility, given the contamination of bird droppings with uric nitrogen, protein nitrogen is measured in faeces (Terpstra method; Terpstra K. D. and N. De Hart. 1973. “The estimation of urinary nitrogen and faecal nitrogen in poultry excreta” Zeitschrift für Tierphysiologie Tierern\ddot{ā}hrung and Futtermittelkunde. 32 (1-5): 306-320).

The true protein digestibility measured as a percentage is therefore calculated according to a quantitative method by the difference between the quantity of ingested hydrolyzate and the quantity of excreted faeces, corrected for endogenous losses based on the following formula.

True protein digestibility %=(ingested protein hydrolyzate−(excreted protein faeces−endogenous excreted protein))/−ingested hydrolyzate protein×100.

The hydrolyzate according to the invention is therefore very easily assimilated by the body.

### Example 3—Raw Material for Animal Feeding

A raw material for animal nutrition is prepared from the hydrolyzate, which composition in free amino acids is presented in Table 3 (2nd column) without additions of additional amino acids, in particular without addition of L-tyrosine, precursor of melanin, the pigment that accounts for the dark colors (brown and black) of the coat and without the addition of L-Cystine, essential for the good health of the skin and constituent of the keratin in the animal fur.

The prepared raw material is anallergic.

In addition, its palatability on cats and dogs has been observed.

1.-11. (canceled)

12. A keratin hydrolyzate comprising at least 88% by weight of free amino acids relative to the total weight of the amino acids of the hydrolyzate, said hydrolyzate comprising free tyrosine in a content ranging from 2 to 4% by weight relative to the total weight of the free hydrolyzate amino acids.

13. The keratin hydrolyzate according to claim 12, wherein the hydrolyzate comprises at least 90% of cystine in free form by weight relative to the total weight of cystine in the hydrolyzate.

14. The keratin hydrolyzate according to claim 12, wherein the hydrolyzate is desalinated, that is it comprises less than 11%, by weight of salts relative to the weight total of the hydrolyzate, the salts being chosen from sodium chloride, sodium sulphate, sodium phosphate, potassium chloride, potassium sulphate and potassium phosphate.

15. The keratin hydrolyzate according to claim 12, wherein the hydrolyzate comprises the following free amino acids:
at least 95% of aspartic acid in free form by weight relative to the total weight of aspartic acid in the hydrolyzate;
at least 95% of threonine in free form by weight relative to the total weight of threonine in the hydrolyzate;
at least 95% of serine in the free form by weight relative to the total weight of serine in the hydrolyzate;
at least 93% of glutamic acid in the free form by weight relative to the total weight of glutamic acid in the hydrolyzate;
at least 90% of glycine in the free form by weight relative to the total weight of glycine in the hydrolyzate;
at least 90% of alanine in the free form by weight relative to the total weight of alanine in the hydrolyzate;
at least 90% of phenylalanine in the free form by weight relative to the total weight of phenylalanine in the hydrolyzate; and
at least 93% of proline in free form by weight relative to the total weight of proline in the hydrolyzate.

16. The keratin hydrolyzate according to claim 12, obtained from a keratin material comprising 17 amino acids, in which, for each of the amino acids, the percentage variation between the weight of the free amino acid in the hydrolyzate and the weight of this amino acid in the starting keratin material is less than 20% in absolute value.

17. The hydrolyzate according to claim 12, wherein the hydrolyzate comprises the following free amino acids, by weight, relative to the total weight of the free amino acids of the hydrolyzate:
aspartic acid in a content ranging from 6.00 to 10.00% by weight;
threonine in a content ranging from 3.00 to 7.00% by weight;
serine in a content ranging from 11.00 to 15.00%, by weight;
glutamic acid in a content ranging from 8.50 to 12.50%, by weight;
glycine in a content ranging from 6.50 to 10.50% by weight;
alanine in a content ranging from 3.00 to 7.00% by weight;
valine in a content ranging from 3.50 to 7.50% by weight;
cystine in a content ranging from 4.00 to 8.00% by weight;
methionine in a content ranging from 0.10 to 2.00% by weight;
isoleucine in a content of 1.50 to 5.50% by weight;
leucine in a content ranging from 6.00 to 10.00% by weight;
tyrosine in a content ranging from 2.50 to 3.50% by weight;
phenylalanine in a content ranging from 3.00 to 7.00% by weight;
lysine in a content ranging from 0.50 to 3.00% by weight;
histidine in a content ranging from 0.10 to 2.00% by weight;
arginine in a content ranging from 4.00 to 8.00% by weight; and
proline in a content ranging from 8.50 to 12.50% by weight.

18. A method for preparing the hydrolyzate according to claim 12, from an animal keratin material, wherein said process comprises at least the following steps, in the order given below:
subjecting the keratin material to at least one chemical hydrolysis by means of an acid under conditions capable of obtaining a hydrolyzate comprising at least 88% by weight of free amino acids relative to the total weight of the amino acids of the hydrolyzate, the rest of the amino acids of the hydrolyzate being in the form of peptides having a molecular mass less than or equal to 800 Dalton;
subjecting the hydrolyzate to a step of adjusting the pH to a value ranging from 3 to 5 and recovering the precipitate and the liquid phase;
separating the precipitate and the liquid phase;
subjecting the precipitate to at least one washing with water until a desalted precipitate is obtained comprising less than 1% of salts by weight relative to the total weight of the precipitate, and recovering the desalted precipitate, on the one hand, and washing waters, on the other one,
combining the washing waters and the liquid phase to obtain a solution and proceed with desalinating this solution by electrodialysis to obtain a desalinated solution,
adding the desalted precipitate to the desalted solution, and
recovering the desalted obtained suspension.

19. The method for preparing the hydrolyzate according to claim 18, in which the chemical hydrolysis is performed for a period ranging from 1 hour to 24 hours at a temperature ranging from 100 to 115° C.

20. The method s for preparing the hydrolyzate according to claim 18, in which the chemical hydrolysis is performed in two stages:
a first chemical hydrolysis performed at a temperature ranging from 60 to 80° C. for 4 to 5 hours followed by
a second chemical hydrolysis performed at a temperature ranging from 100 to 115° C. for 5 to 8 hours.

21. The method for preparing the hydrolyzate according to claim 18, in which the obtained suspension is dried and the solid obtained at the end of the drying recovered.

22. A product comprising the hydrolyzate according to claim 12, wherein product chosen from foodstuffs for animal feed for pets, feed for aquaculture, and plant biostimulants.

23. The hydrolyzate according to claim 17, wherein the hydrolyzate comprises the following free amino acids, by weight, relative to the total weight of the free amino acids of the hydrolyzate:
aspartic acid in a content ranging from 7.00 to 9.00% by weight;
threonine in a content ranging from 4.00 to 6.00% by weight;
serine in a content ranging from 12.00 to 14.00% by weight;
glutamic acid in a content ranging from 9.50 to 11.50% by weight;
glycine in a content ranging from 7.50 to 9.50% by weight;
alanine in a content ranging from 4.00 to 6.00% by weight;
valine in a content ranging from 4.50 to 6.50% by weight;
cystine in a content ranging from 5.00 to 7.00% by weight;
methionine in a content ranging from 0.20 to 1.00% by weight;
isoleucine in a content ranging from 2.50 to 4.50% by weight;
leucine in a content ranging from 7.00 to 9.00% by weight;
tyrosine in a content ranging 3.00 to 3.50% by weight;
phenylalanine in a content ranging from 4.00 to 6.00% by weight;
lysine in a content ranging from 1.00 to 2.00% by weight;
histidine in a content ranging from 0.20 to 1.00% by weight;
arginine in a content ranging from 5.00 to 7.00% by weight; and
proline in a content ranging from 9.50 to 11.50% by weight.
